(19) United States
(12) Patent Application Publication
(10) Pub. No.: US 2004/0241115 A1
(43) Pub. Date: Dec. 2, 2004

(54) Nail varnish composition comprising fibers covered with at least one fluorescent whitening agent

(76) Inventor: Roland Ramin, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/801,848

(22) Filed: Mar. 17, 2004

Related U.S. Application Data
(60) Provisional application No. 60/459,645, filed on Apr. 3, 2003.

(30) Foreign Application Priority Data
Mar. 19, 2003 (FR) 03 03355

(51) Int. Cl. A61K 7/04

(52) U.S. Cl. 424/61

(57) ABSTRACT

A nail varnish composition comprising, in a cosmetically acceptable medium, fibers covered with at least one fluorescent whitening agent. A cosmetic process for making up and/or for the non-therapeutic care of nails comprising applying to the nails the nail varnish composition. Also disclosed is a method for conferring on a coating obtained after application of the nail varnish composition to the nails at least one property chosen from providing a homogeneous and even appearance, concealing nail imperfections, providing good hold and mechanical strength, and providing a nail-strengthening effect.

This application claims benefit of U.S. Provisional Application No. 60/459,645, filed Apr. 3, 2003, which is herein incorporated by reference.

Disclosed herein is a nail varnish composition comprising, in a cosmetically acceptable medium, fibers covered with at least one fluorescent whitening agent. Further disclosed is a process for making up and/or caring for the nails.

The composition can be employed as a product for making up the nails, such as a colored nail varnish; as a base coat for the nails; as a top coat, to be applied under or over the product for making up the nails; or as a product for the cosmetic care of the nails, such as a treating base intended to protect, to strengthen and/or to repair the nails.

This composition can be applied to the nails of human beings or to false nails. For example, the composition disclosed herein may be a treating base for nail varnishes.

It is known by formulators to use fibers in nail varnishes, for example, for repairing and strengthening broken nails, as described in Patent No. FR-A-1 529 329. Further, for example, Patent No. EP 0 583 347 B1 discloses nail varnishes comprising aramide fibers which may make it possible to obtain good adhesion of the varnish to the nail. Patent Application Publication No. WO-A-02/41852 also discloses a nail varnish having a velvety feel and a glossy visual appearance by virtue of the use of flat fibers.

The use of fibers in nail varnish compositions, however, may often be characterized by poor dispersion of the fibers, which is reflected by a non-homogeneous coating on the nail with a non-smooth and granular appearance.

This is why there is a desire to obtain a nail varnish composition comprising fibers that may be capable of strengthening the nails while also making it possible to obtain a film with a visual appearance that is satisfactory to the user.

The present inventor has found that a nail varnish composition comprising fibers covered with at least one fluorescent whitening agent may make it possible to obtain at least one of properties described above.

Such a composition may make it possible to obtain, after application to the nails, a coating with a homogeneous and even appearance which conceals the imperfections of the nails (for example, scratched, cracked or split nails) by virtue of the fibers coated with the at least one fluorescent whitening agent and may confer a healthy appearance thereon. The film deposited on the nail may exhibit good properties of hold and of mechanical strength; for example, it may withstand being rubbed, impacted or scratched.

Thus, disclosed herein is a nail varnish composition comprising, in a physiologically acceptable medium, fibers covered with at least one fluorescent whitening agent.

The use of a fluorescent whitening agent in nail varnish compositions is known, for example from Patent No. FR-A-2 741 261, which discloses cosmetic compositions comprising a brightening fluorescent agent, also known as a fluorescent whitening agent, for increasing the apparent intensity of the color of a nail varnish. Patent No. FR-A-2 773 470 also provides for the use of a fluorescent additive in products for making up the nails which may make it possible to neutralize the yellow effect given by resins or which may exhibit a fluorescent blue glint under illumination.

None of these documents, however, disclose or suggest a composition comprising fibers covered with at least one fluorescent whitening agent such as the composition disclosed herein.

Further disclosed herein is a cosmetic process for making up and/or for the non-therapeutic care of nails, comprising applying, to the nails, at least one layer of a nail varnish composition comprising, in a physiologically acceptable medium, fibers covered with at least one fluorescent whitening agent.

Further disclosed herein is a method for forming a coating, obtained after application of a nail varnish composition to the nails, having at least one property chosen from providing a homogeneous and even appearance, concealing nail imperfections, providing good hold and mechanical strength of the coating, and providing a nail-strengthening effect. The method according to this aspect comprises including in the nail varnish composition, comprising a cosmetically acceptable medium, fibers covered with at least one fluorescent whitening agent, applying the nail varnish composition to the nail, and forming a coating on the nail.

As used herein, the term “cosmetically acceptable medium” means a medium compatible with making up the nails.

As used herein, the term “fiber” means an object with a length L and with a diameter D such that L is much greater than D, wherein D is the diameter of the circle in which the cross section of the fiber is inscribed. For example, the ratio L/D (or shape factor) may range from 3.5:1 to 2,500:1, further, for example, from 5:1 to 500:1 and, even further, for example, from 5:1 to 150:1.

The fibers used in the composition disclosed herein are chosen from commercially available fibers which may already be covered with the at least one fluorescent whitening agent and fibers which are not covered with the at least one fluorescent whitening agent and which are subsequently covered with the at least one fluorescent whitening agent by processes known to a person skilled in the art.

The fibers which can be used in the composition disclosed herein may be chosen from fibers of synthetic and natural origin and fibers of inorganic and organic origin. The fibers may be chosen from short and long fibers; individual and organized fibers, for example braided; and hollow and solid fibers. The fibers can have any shape and may, for example, have a circular or polygonal (square, hexagonal or octagonal) cross section, according to the specific application envisaged. For example, the ends of the fibers may be blunted and/or smoothed in order to avoid inflicting injury.

For example, the fibers may have a length ranging from 1 nm to 10 mm. Examples of such fibers include nanometric fibers having a length ranging from 1 nm to 999 nm. In addition, the fibers may have a length ranging from 1 μm to 10 mm, further, for example, from 0.1 mm to 5 mm and, even further, for example, from 0.3 mm to 1 mm. The fibers' cross section may be inscribed within a circle with a diameter ranging from 2 nm to 500 m, for example, ranging from 100 nm to 100 m and, further, for example, from 1 μm to 50 μm. The weight or count of the fibers is often given in denier or decitex and represents the weight in grams per 9 km of yarn. For example, the fibers disclosed herein may have a count ranging from 0.01 to 10 denier, further, for example, from 0.1 to 2 denier and, even further, for example, from 0.3 to 0.7 denier.

The fibers can be chosen from those used in the manufacture of textiles and, for example, at least one of fibers of silk; cotton; wool; flax; cellulose, for example, extracted, such as from wood, vegetables and algae; rayon; polyamide (NYLON); viscose, acetate, for example, rayon acetate; poly(p-phenylene terephthalamide) (and aramide), for example, KEVLAR; acrylic polymer, for example, poly(methyl methacrylate) and poly(2-hydroxyethyl methacrylate); polyolefin, for example, polyethylene and polypropylene; glass; silica; carbon, for example, in graphite form; polytetrafluoroethylene (such as TEFLON); insoluble collagen; polyesters; poly(vinyl chloride); poly(vinylidene chloride); poly(vinyl alcohol); polyacrylonitrile; chitosan; polyurethane and poly(ethylene phthalate); and fibers formed from a blend of polymers such as those mentioned above, for example polyamide/polyester fibers.

For example, the fibers may be polyamide (NYLON) fibers.

Further examples of the fibers disclosed herein include fibres used in surgery, such as resorbable synthetic fibers prepared from glycolic acid and caprolactone (MONOCRYL from Johnson & Johnson); resorbable synthetic fibers of the copolymer of lactic acid and of glycolic acid (VICRYI from Johnson & Johnson); poly(terephthalic ester) fibers (ETHIBOND from Johnson & Johnson); and stainless steel wires (STEEL WIRE from Johnson & Johnson).

Furthermore, in addition to their coating of the at least one fluorescent whitening agent, the fibers may or may not be surface-treated and/or may or may not be coated with a protective layer. This surface treatment and/or coating lies under the coating of the at least one fluorescent whitening agent if the constituent material of the protective layer is opaque or semi-opaque; it can lie above the coating of the at least one fluorescent whitening agent if this material is transparent or semi-transparent.

For example, the coated fibers which can be used in the composition disclosed herein may be chosen from polyamide fibers coated with copper sulphide for an antistatic effect (for example, R-STAT from Rhodia) and another polymer which makes possible a particular arrangement of the fibers (specific surface treatment). Further examples of the coated fibers include fibers coated with inorganic or organic pigments, such as the pigments disclosed herein.

For example, the fibers may be chosen from fibers of synthetic origin and, for example, organic fibers, such as those used in surgery.

The fibers which can be used in the composition disclosed herein may also be chosen from polyamide, cellulose, poly(p-phenylene terephthalamide) and polyethylene fibers. Their length (L) can range from 0.1 mm to 5 mm, for example, from 0.25 mm to 1.6 mm, and their mean diameter can range from 1 μm to 50 μm. For example, the polyamide fibers sold by Etablissements P. Bonte under the name “Polyamide 0.9 Dtex 3 mm”, having a mean diameter of 6 μm, a count of approximately 0.9 dtex and a length ranging from 0.3 mm to 5 mm may be used in an aspect disclosed herein. In another aspect, cellulose fibers (or rayon fibers) having a mean diameter of 50 μm and a length ranging from 0.5 mm to 6 mm, such as those sold under the name “Natural rayon flock fiber RC1 BE-N003-M04” by Claremont Flock may be used. Further examples of fibers that may be used include polyethylene fibers, such as those sold under the name of SHURT STUFF 13 099 F by Mini Fibers.

For example, fibers covered with at least one fluorescent whitening agent as sold by LCW under the trade name Fiberlon 54 Z03, having a length of approximately 0.4 mm and a count of 0.5 denier may be used.

The composition disclosed herein can also comprise “rigid” fibers, substantially rectilinear, in contrast to the fibers mentioned above, which are not rigid necessarily fibers.

Rigid fibers, which are initially substantially straight, do not undergo a substantial modification in their shape when they are placed in a dispersing medium. This is reflected by the angular condition defined below, reflecting a shape which may be described as always substantially straight or linear. This angular condition reflects the stiffness of the fibers, which can be difficult to express by another parameter for objects as small as the rigid fibers.

The stiffness of the fibers is reflected by the following angular conditions: For example, at least 50% by number, further, for example, at least 75% by number and, even further, for example, at least 90% by number of the fibers are such that the angle formed between the tangent to the central longitudinal axis of the fiber and the straight line connecting one of the ends to the point on the central longitudinal axis of the fiber corresponding to half the length of the fiber is less than 150 and the angle formed between the tangent to the central longitudinal axis of the fiber at a point situated midway along the fiber and the straight line connecting one of the ends to the point on the central longitudinal axis of the fiber corresponding to half the length of the fiber is less than or equal to 150, for the same fiber length ranging from 0.8 mm to 5 mm, for example, ranging from 1 mm to 4 mm, further, for example, ranging from 1 mm to 3 mm and, even further, for example, a fiber length of 2 mm.

The angle mentioned above can be measured at the two ends of the fiber and at a point situated midway along the fiber. In this case, three measurements are made and the mean of the angles measured is less than or equal to 150. In other words, the tangent, at any point on the fiber, forms an angle of less than 15°.

As used herein, the angle formed by the tangent at a point on the fiber is the angle formed between the tangent to the central longitudinal axis of the fiber at the point on the fiber and the straight line connecting the end of the fiber closest to the point on the central longitudinal axis of the fiber corresponding to half the length of the fiber.

Generally, the rigid fibers which can be used in the composition disclosed herein have the same fiber length or a substantially similar length.

For example, when a medium in which the rigid fibers are dispersed at a concentration of the fibers of 1% by weight is observed under a microscope, with an objective lens allowing a magnification of 2.5 and with a full field view, most of the rigid fibers, that is to say at least 50% by number of the rigid fibers, for example, at least 75% by number of the rigid fibers and, further, for example, at least 90% by number of the rigid fibers, meet the angular condition defined above. The measurement resulting in the value of the angle is carried out for the same length of fibers; this length may range from 0.8 mm to 5 mm, for example, from 1 to 4 mm, for example, from 1 to 3 mm, and may, for example, be 2 mm.

The medium in which the observation is carried out is a dispersing medium which ensures good dispersion of the rigid fibers, for example, water or an aqueous gel of clay or of associative polyurethane. Direct observation of the composition comprising the rigid fibers can even be carried out. A sample of the composition or of the dispersion prepared is placed between a slide and a cover slip for observation under a microscope with an objective lens allowing a magnification of 2.5 and with a full field view. The full field view makes it possible to see the fibers in their entirety.

The rigid fibers can be chosen from fibers of a synthetic polymer, wherein the synthetic polymer is chosen from polyesters, polyurethanes, acrylic polymers, polyolefins, polyamides, for example, non-aromatic polyamides, and aromatic polyimideamides.

Examples of rigid fibers include:

polyester fibers, such as those obtained by cutting up yarns sold under the names Fiber 255-100-R11-242T Size 3 mm (octalobed cross section), Fiber 265-34-R1′-56T Size 3 mm (round cross section) and Fiber Coolmax 50-34-591 Size 3 mm (tetralobed cross section) by Dupont de Nemours; -polyamide fibers, such as those sold under the names TRILOBAL NYLON 0.120-1.8 DPF; TRILOBAL NYLON 0.120-18 DPF; and NYLON 0.120-6 DPF by Cellusuede Products; and obtained by cutting up yarns sold under the name FIBER NOMEX Brand 430 Size 3 mm by Dupont de Nemours;

polyimideamide fibers, such as those sold under the name KERMEL and KERMEL TECH by Rhodia;

poly(p-phenylene terephthalamide) fibers (or aramide fibers), sold, for example, under the name KEVLAR by Dupont de Nemours; and

fibers with a multiplayer structure comprising alternating layers of polymers chosen from polyesters, acrylic polymers and polyamides, such as those disclosed in Patent Nos. EP-A-6 921 217, EP-A-686 858 and U.S. Pat. No. 5,472,798. Such fibers are sold under the names MORPHOTEX and TEIJIN TETRON MORPHOTEX by Teijin.

The rigid fibers may, for example, be aromatic polyimideamide fibers.

Polyimideamide yarns or fibers which can be used for the compositions disclosed herein are described, for example, in the document by R. Pigeon and P. Allard, Chimie Macromoleculaire Appliquée, 40/41 (1974), pages 139-158 (No. 600), and in Patent Nos. U.S. Pat. No. 3,802,841, FR-A-2 079 785, EP-A1-0 360 728 or EP-A-0 549 494, to which reference relating to the polyimideamide yarns or fibers may be made, which are herein incorporated by reference. [Q40] For example, the aromatic polyimideamide fibers may be polyimideamide fibers comprising repeat units of formula:

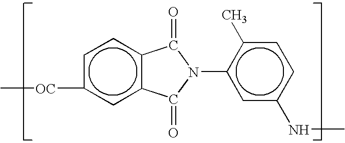

obtained by polycondensation of toluylene diisocyanate and of trimellitic anhydride.

The fibers can be present in the composition disclosed herein in an amount ranging from 0.1% to 10% by weight, for example, from 0.5% to 5% by weight and, further, for example, from 1% to 3% by weight, relative to the total weight of the composition.

The fibers used in the composition disclosed herein are covered with at least one fluorescent whitening agent.

Fluorescent whitening agents are compounds well known to a person skilled in the art. Such agents are described in “Fluorescent Whitening Agent, Encyclopedia of Chemical Technology, Kirk-Othmer”, vol. 11, p. 227-241, 4^{th} edition, 1994, Wiley and the disclosure relating to such fluorescent whitening agents is specifically incorporated herein by reference.

The at least one fluorescent whitening agent can be defined, for example, as compounds which absorb essentially in the UV-A region ranging from 300 to 390 nm and re-emit in the region ranging from 400 to 525 nm.

The at least one fluorescent whitening agent may be chosen from stilbene derivatives, for example, polystyrylstilbenes and triazinylstilbenes; coumarin derivatives, for example, hydroxycoumarins and aminocoumarins; oxazole, benzoxazole, imidazole, triazole and pyrazoline derivatives; pyrene derivatives; and porphyrin derivatives.

Such compounds are readily available commercially. Examples of such compounds include:

naphthotriazole stilbene derivatives sold under the trade name TINOPAL GS, disodium 4,4′-distyrylbiphenyl disulphonate (CTFA name: disodium distyrylbiphenyl disulfonate), sold under the trade name TINOPAL CBS-X, the cationic aminocoumarin derivative sold under the trade name TINOPAL SWN Conc., sodium 4,4′-bis[(4,6-dianilino-1,3,5-triazin-2-yl)amino]stilbene-2,2′-disulphonate, sold under the trade name TINOPAL SOP, 4,4′-bis[(4-anilino-6-bis(2-hydroxyethyl)amino-1,3,5-triazin-2-yl)amino]stilbene-2,2′-disulphonic acid, sold under the trade name TINOPAL UNPA-GX, 4,4′-bis[(4-anilino-6-morpholino-1,3,5-triazin-2-yl)amino]stilbene, sold under the trade name TINOPAL AMS-GX, and disodium 4,4′-bis[(4-anilino-6-(2-hydroxyethyl)methylamino-1,3,5-triazin-2-yl)amino]stilbene-2,2′-disulphonate, sold under the trade name TINOPAL I 5BM-GX, all by Ciba Specialty Chemicals,

2,5-thiophenediylbis(5-tert-butyl-1,3-benzoxazole), sold under the trade name UVITEX OB by Ciba,

anionic derivative of diaminostilbene as a dispersion in water, sold under the trade name LEUCOPHOR BSB Liquid by Clariant, and

fluorescent whitening agent lakes sold under the trade name COVAZUR by Wackherr.

The at least one fluorescent whitening agent which can be used in the composition disclosed herein can also be provided in the form of copolymers, for example of acrylates and/or of methacrylates, grafted by fluorescent whitening agent groups, as disclosed in Patent Application No. FR 99/10942.

The at least one fluorescent whitening agent may, for example, be chosen from disodium 4,4′-distyrylbiphenylsulphonate, sodium 4,4′-bis[(4,6-dianilino-1,3,5-triazin-2-yl)amino]stilbene-2,2′-disulphonate, and 2,5-thiophenediylbis(5-tert-butyl-1,3-benzoxazole).

The composition disclosed herein can comprise an organic solvent medium, comprising at least one organic solvent, or an aqueous medium.

Examples of the at least one organic solvent which can be used in the composition disclosed herein include:

ketones which are liquid at ambient temperature, such as methyl ethyl ketone, methyl isobutyl ketone, diisobutyl ketone, isophorone, cyclohexanone and acetone;

alcohols which are liquid at ambient temperature, such as ethanol, isopropanol, diacetone alcohol, 2-butoxyethanol and cyclohexanol;

glycols which are liquid at ambient temperature, such as ethylene glycol, propylene glycol, pentylene glycol and glycerol;

propylene glycol ethers which are liquid at ambient temperature, such as propylene glycol monomethyl ether, propylene glycol monomethyl ether acetate and dipropylene glycol mono(n-butyl) ether;

short-chain esters (comprising a total of 3 to 8 carbon atoms), such as ethyl acetate, methyl acetate, propyl acetate, n-butyl acetate and isopentyl acetate;

alkanes which are liquid at ambient temperature, such as decane, heptane, dodecane and cyclohexane; and

aldehydes which are liquid at ambient temperature, such as benzaldehyde and acetaldehyde.

The at least one organic solvent may, for example, be chosen from short-chain esters (comprising a total of 3 to 8 carbon atoms) and alcohols which are liquid at ambient temperature.

The composition with an organic solvent medium can additionally comprise water, for example, in an amount ranging from 0.1 to 10% by weight, relative to the total weight of the composition.

When the nail varnish composition comprises an organic solvent medium, the at least one organic solvent may be present in an amount ranging from 5% to 95% by weight, for example, from 50% to 70% by weight, relative to the total weight of the composition.

When the nail varnish composition comprises an aqueous medium, the aqueous medium can comprise water (for example, the aqueous medium may be water) or can comprise a mixture of water and at least one water-miscible solvent, such as lower monoalcohols comprising from 1 to 5 carbon atoms, for example, ethanol, glycols comprising from 2 to 8 carbon atoms, C₃-C₄ ketones and C₂-C₄ aldehydes.

The water content of the composition can range from 5 to 95% by weight, for example, from 50 to 70% by weight, relative to the total weight of the composition.

The composition can further comprise at least one film-forming polymer in an amount ranging from 1 to 70% by weight, for example, from 10 to 45% by weight, relative to the total weight of the composition, in an organic medium or an aqueous medium as defined above.

As used herein, the term “film-forming polymer” means a polymer capable of forming, by itself alone or in the presence of a possible plasticizer, an isolable film. The at least one film-forming polymer can be dissolved or dispersed in the form of particles in the cosmetically acceptable medium of the composition.

The at least one film-forming polymer can be chosen from radical polymers, polycondensates and polymers of natural origin.

The at least one film-forming polymer can be chosen, for example, from vinyl and acrylic polymers, polyurethanes, polyesters, alkyd resins, epoxy ester resins, cellulose polymers, such as nitrocellulose, cellulose esters, such as cellulose acetate, cellulose acetate propionate and cellulose acetate butyrate, the resins resulting from the condensation of formaldehyde with an arylsulphonamide.

When the composition comprises an organic medium, the at least one film-forming polymer can be chosen, for example, from alkyd, acrylic resins, vinyl resins, polyurethanes and polyesters, celluloses and cellulose derivatives, such as nitrocellulose and cellulose esters, such as cellulose acetate, cellulose acetate propionate and cellulose acetate butyrate, the resins resulting from the condensation of formaldehyde with an arylsulphonamide.

When the composition comprises the aqueous medium, then the at least one film-forming polymer may be present in the form of particles in dispersion in the aqueous medium, thus forming a latex or pseudolatex.

The film-forming polymers capable of being used in the aqueous medium may be chosen from at least one of polyurethanes, for example, anionic polyurethanes, polyester-polyurethanes, polyether-polyurethanes, radical polymers, for example, acrylic, acrylic/styrene and vinyl radical polymers, polyesters, and alkyd resins.

The dispersion can also comprise at least one associative polymer chosen from polyurethanes and natural gums, such as xanthan gum.

Examples of polymers in aqueous dispersions include the dispersions of acrylic polymers sold under the names NEOCRYL XK-90, NEOCRYL A-1070, NEOCRYL A-1090, NEOCRYL BT-62, NEOCRYL A-1079 and NEOCRYL A-523 by Zeneca and DOW LATEX 432® by Dow Chemical. The composition disclosed herein may also comprise aqueous polyurethane dispersions, for example, the polyester-polyurethanes sold under the names AVALURE UR-405, AVALURE UR-410, AVALURE UR-425 and SANCURE 2060 by Goodrich and the polyether-polyurethanes sold under the names SANCURE 878 by Goodrich and NEOREZ R-970 by Avecia.

The at least one film-forming polymer can be present in the composition disclosed herein in an amount on a dry basis ranging from 1% to 70% by weight, for example, from 10% to 45% by weight, relative to the total weight of the composition.

At least one additional agent which is able to form a film can be provided in order to improve the film-forming properties of the nail varnish composition.

The at least one additional agent which is able to form a film can be chosen from any compounds known to a person skilled in the art as being capable of fulfilling the desired function and can be chosen, for example, from plasticizers.

In addition, when the composition disclosed herein comprises at least one film-forming polymer in the form of particles dispersed in an aqueous medium, the at least one additional agent which is able to form a film can also be chosen from coalescents.

The composition can additionally comprise at least one plasticizer. For example, the at least one plasticizer can be chosen from conventional plasticizers, such as:

glycols and derivatives thereof, such as diethylene glycol ethyl ether, diethylene glycol methyl ether, diethylene glycol butyl ether, diethylene glycol hexyl ether, ethylene glycol ethyl ether, ethylene glycol butyl ether and ethylene glycol hexyl ether;

glycol esters;

propylene glycol derivatives and, for example, propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether;

esters of acids, for example, carboxylic acids, such as citrates, phthalates, adipates, carbonates, tartrates, phosphates and sebacates; and

oxyethylenated derivatives, such as oxyethylenated oils, for example, vegetable oils, such as castor oil.

The amount of the at least one plasticizer can be chosen by a person skilled in the art on the basis of his or her general knowledge, so as to obtain a composition having cosmetically acceptable properties. The content of at least one plasticizer can, for example, range from 0.1% to 15% by weight and, for example, from 0.5% to 10% by weight, relative to the total weight of the composition.

The composition can comprise at least one coloring material which can be chosen from pulverulent compounds and dyes which are soluble in the medium of the composition. The at least one coloring material can be present in an amount ranging from 0.01% to 10% by weight, relative to the total weight of the composition.

The pulverulent compounds can be chosen from at least one of pigments, pearlescent agents, and glitter generally used in nail varnishes.

The pigments can be chosen from at least one of white and colored and inorganic and organic pigments. Examples of inorganic pigments include titanium dioxide, which has optionally been surface-treated, zirconium, cerium, iron and chromium oxides, manganese violet, ultramarine blue, chromium hydrate, ferric blue and metal pigments, such as aluminium, copper and bronze. Examples of organic pigments include carbon black, D & C pigments, lakes based on cochineal carmine and on barium, strontium, calcium and aluminium, and guanine.

The pigments may also be chosen from pigments with an effect, such as at least one of particles comprising an organic and inorganic and natural and synthetic substrate, for example glass, acrylic, polyester, polyurethane and poly(ethylene terephthalate) resins; ceramics and aluminas, which substrate may or may not be covered with metal substances, such as aluminium, gold, copper and bronze, and with metal oxides, such as titanium dioxide, iron oxide and chromium oxide, and with inorganic or organic pigments.

The pearlescent pigments can be chosen from white pearlescent pigments, such as mica covered with titanium oxide and with bismuth oxychloride; colored pearlescent pigments, such as titanium oxide-coated mica with iron oxides; titanium oxide-coated mica with, for example, ferric blue or chromium oxide; titanium oxide-coated mica with at least one organic pigment of the abovementioned type; and fluorophlogopites with iron oxides.

The pigments may also be chosen from pigments with goniochromatic properties, for example, liquid crystal and multiplayer pigments.

The dyes may, for example, be chosen from Sudan red, DC Red 17, DC Green 6, p-carotene, soybean oil, Sudan brown, DC Yellow 11, DC Violet 2, DC Orange 5 and quinoline yellow.

The at least one coloring material can also be chosen from the abovementioned fluorescent whitening agents.

The composition disclosed herein can comprise, in addition to the fibers coated with the at least one fluorescent whitening agent, fibers which are not coated with the at least one fluorescent whitening agent.

The composition disclosed herein can additionally comprise at least one other ingredient or additive commonly used in cosmetic compositions and known to a person skilled in the art as being capable of being incorporated in a nail varnish composition.

The at least one ingredient or additive can be chosen from coalescents, thickening agents, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, and the like.

Of course, a person skilled in the art will take care to choose this or these possible additional compound(s) and/or the amount(s) so that the properties of the composition disclosed herein are not, or not substantially, detrimentally affected by the envisaged addition(s).

The examples which follow illustrate the various embodiments disclosed herein in a non-limiting way.

### EXAMPLE 1

### Nail Varnish

A nail varnish was prepared which had the following composition:

| | | |
|---|---|---|
| Nitrocellulose | 10 | g |
| Tributyl citrate | 5 | g |
| Alkyd resin | 10 | g |
| Pigments | 0.2 | g |
| Thickening agent | 1.5 | g |
| Fibers covered with fluorescent whitening agent | | |
| (Fiberlon 54 ZO3 from LCW) | 5 | g |
| Isopropyl alcohol | 5 | g |
| Ethyl acetate | 20 | g |
| Butyl acetate | q.s. for 100 | g |

Procedure:

The nitrocellulose was dissolved in the solvent mixture (ethyl acetate and butyl acetate) and then the plasticizer (tributyl citrate) and the alkyd resin were added to the combination.

The thickening agent and the pigments were dispersed and milled using a triple roll mill, either in all or in a portion of the above mixture.

The fibers were added and dispersed in the mixture with vigorous stirring.

After application to the nails, the varnish film was assessed as exhibiting good properties of hold and of resistance to impacts and rubbing and having a repairing effect on the nail.

### EXAMPLE 2

### Treating Base Composition

A nail varnish transparent treating base was prepared which had the following composition:

| | | |
|---|---|---|
| Nitrocellulose | 10 | g |
| Tributyl acetylcitrate | 5 | g |
| Alkyd resin | 10 | g |
| Pyrogenic silica | 1.5 | g |
| Fibers covered with fluorescent whitening agent | 2 | g |
| (Fiberlon 54 ZO3 from LCW) | | |
| Isopropyl alcohol | 5 | g |
| Ethyl acetate | 20 | g |
| Butyl acetate | q.s. for 100 | g |

Procedure:

The nitrocellulose was dissolved in the solvent mixture (ethyl acetate and butyl acetate) and then the plasticizer (tributyl acetylcitrate) and the alkyd resin were added to the combination.

The thickening agent and the pigments were dispersed and milled using a triple roll mill, either in all or in a portion of the above mixture.

The fibers were added and dispersed in the mixture with vigorous stirring.

After application of the treating base to the nails, the film was assessed as exhibiting good properties of hold and as conferring, on the nails, a strengthening and repairing effect and can be regarded as a “dressing” for the nail.

After application to damaged nails, this treating base was assessed as conferring a healthy appearance on these nails.

What is claimed is:

1. A nail varnish composition comprising, in a cosmetically acceptable medium, fibers covered with at least one fluorescent whitening agent.

2. The nail varnish composition according to claim 1, wherein the fibers are chosen from at least one of fibers of silk, cotton, wool, flax, cellulose, rayon, polyamide, viscose, acetate, poly(p-phenylene terephthalamide), poly(p-phenylene terephthalaramide), acrylic, polyolefin, glass, silica, carbon, polytetrafluoroethylene, insoluble collagen, polyesters, poly(vinyl chloride), poly(vinylidene chloride), poly(vinyl alcohol), polyacrylonitrile, chitosan, polyurethane and poly(ethylene phthalate), fibers formed from blends of polymers, and substantially rectilinear rigid fibers.

3. The nail varnish composition according to claim 2, wherein the acetate fibers are rayon acetate fibers.

4. The nail varnish composition according to claim 2, wherein the acrylic fibers are chosen from poly(methyl methacrylate) and poly(2-hydroxyethyl methacrylate) fibers.

5. The nail varnish composition according to claim 2, wherein the polyolefin fibers are chosen from polyethylene and polypropylene fibers.

6. The nail varnish composition according to claim 2, wherein the carbon fibers are graphite carbon fibers.

7. The nail varnish composition according to claim 1, wherein the fibers are fibers of synthetic origin.

8. The nail varnish composition according to claim 1, wherein the fibers have a length ranging from 1 nm to 10 mm.

9. The nail varnish composition according to claim 8, wherein the fibers have a length ranging from 1 μm to 10 mm.

10. The nail varnish composition according to claim 9, wherein the fibers have a length ranging from 0.1 mm to 5 mm.

11. The nail varnish composition according to claim 10, wherein the fibers have a length ranging from 0.3 mm to 1 mm.

12. The nail varnish composition according to claim 1, wherein the fibers have a cross section inscribed within a circle with a diameter ranging from 2 nm to 500 μm.

13. The nail varnish composition according to claim 12, wherein the fibers have a cross section inscribed within a circle with a diameter ranging from 100 nm to 100 μm.

14. The nail varnish composition according to claim 1, wherein the fibers are present in an amount ranging from 0.1% to 10% by weight, relative to the total weight of the composition.

15. The nail varnish composition according to claim 14, wherein the fibers are present in an amount ranging from 0.5% to 5% by weight, relative to the total weight of the composition.

16. The nail varnish composition according to claim 1, wherein the at least one fluorescent whitening agent is chosen from stilbene derivatives, coumarin derivatives, oxazole, benzoxazole, imidazole, triazole and pyrazoline derivatives, pyrene derivatives and porphyrin derivatives.

17. The nail varnish composition according to claim 1, wherein the at least one fluorescent whitening agent is chosen from disodium 4,4′-distyrylbiphenyl disulphonate, sodium 4,4′-bis[(4,6-dianilino-1,3,5-triazin-2-yl)amino]stilbene-2,2′-disulphonate, and 2,5-thiophenediylbis(5-tert-butyl-1,3-benzoxazole).

18. The nail varnish composition according to claim 1, further comprising at least one film-forming polymer.

19. The nail varnish composition according to claim 18, wherein the at least one film-forming polymer is present in an amount ranging from 1 to 70% by weight, relative to the total weight of the composition.

20. The nail varnish composition according to claim 19, wherein the at least one film-forming polymer is present in an amount ranging from 10 to 0.45% by weight, relative to the total weight of the composition.

21. The nail varnish composition according to claim 1., wherein the cosmetically acceptable medium is an organic medium comprising at least one organic solvent.

22. The nail varnish composition according to claim 21, wherein the at least one organic solvent is present in an amount ranging from 5% to 95% by weight, relative to the total weight of the composition.

23. The nail varnish composition according to claim 22, wherein the at least one organic solvent is present in an amount ranging from 50% to 70% by weight, relative to the total weight of the composition.

24. The nail varnish composition according to claim 1, wherein the cosmetically acceptable medium is an aqueous medium.

25. The nail varnish composition according to claim 24, wherein the aqueous medium is present in an amount ranging from 5 to 95% by weight, relative to the total weight of the composition.

26. The nail varnish composition according to claim 25, wherein the aqueous medium is present in an amount ranging from 50 to 70% by weight, relative to the total weight of the composition.

27. The nail varnish composition according to claim 1, further comprising at least one coloring material.

28. The nail varnish composition according to claim 1, wherein the at least one coloring material is present in an amount ranging from 0.01% to 10% by weight, relative to the total weight of the composition.

29. A cosmetic process for making up and/or for the non-therapeutic care of nails, comprising applying, to the nails, at least one layer of a nail varnish composition comprising, in a cosmetically acceptable medium, fibers covered with at least one fluorescent whitening agent.

30. A cosmetic method comprising, including in a nail varnish composition, comprising a cosmetically acceptable medium, fibers covered with at least one fluorescent whitening agent, applying the nail varnish composition to a nail, and forming a coating on the nail, wherein the coating formed has at least one property chosen from providing a homogeneous and even appearance, concealing nail imperfections, providing good hold and mechanical strength, and providing a nail-strengthening effect.
